(19) United States
(12) Patent Application Publication
(10) Pub. No.: US 2025/0295723 A1
(43) Pub. Date: Sep. 25, 2025

(54) CHINESE MEDICINAL COMPOSITION AND USE OF THE SAME FOR TREATING DEMENTIA

(71) Applicant: LI KANG BIOTECHNICAL CO., LTD., Tainan City (TW)

(72) Inventors: Yi-Chang SU, Taipei City (TW); Wen-Fei CHIOU, Taipei City (TW); Young-Ji SHIAO, Taipei City (TW); Yuh-Chiang SHEN, Taipei City (TW); Wen-Chi WEI, Taipei City (TW); Keng-Chang TSAI, Taipei City (TW); Chia-Ching LIAO, Taipei City (TW); Yu-Chi LIN, Taipei City (TW); Yi-Chia HUANG, Taipei City (TW); Jui-Shan LIN, Taipei City (TW); Chien-Jung LIN, Taipei City (TW); Tsung-Yun HSIEH, Taipei City (TW); Yueh-Feng SUNG, Taipei City (TW); Jiunn-Tay LEE, Taipei City (TW)

(21) Appl. No.: 19/085,696

(22) Filed: Mar. 20, 2025

Related U.S. Application Data
(60) Provisional application No. 63/567,958, filed on Mar. 21, 2024.

(51) Int. Cl. A61K 36/539; A61K 35/32; A61K 36/236; A61K 36/48; A61K 36/714; A61K 36/8965; A61P 25/28

(52) U.S. Cl. CPC A61K 36/539; A61K 35/32; A61K 36/236; A61K 36/48; A61K 36/714; A61K 36/8965; A61P 25/28

(57) ABSTRACT

The present invention discloses a Chinese medicinal composition, which Includes: Hedysari Radix, Scutellariae Radix, Asparagi Radix, and Chuanxiong Rhizoma. The present invention also discloses a method for treating dementia, which includes administering to a subject in need thereof the Chinese medicinal composition.

### CROSS-REFERENCE TO RELATED APPLICATION

This application claims the benefit of U.S. Provisional Patent Application No. 63/567,958, filed on Mar. 21, 2024, the entire disclosure of which is incorporated by reference herein.

### TECHNICAL FIELD

The present invention relates to a Chinese medicinal composition including: Hedysari Radix, Scutellariae Radix, Asparagi Radix, and Chuanxiong Rhizoma. The present invention also relates to use of the Chinese medicinal composition for treating dementia.

### PRIOR ART

Based on the cause, dementia can be divided into degenerative dementia caused by neurodegeneration, vascular dementia caused by cerebral vascular obstruction or disease, and mixed dementia in which these two causes coexist. Alzheimer's disease (AD) is the most common among these types of dementias.

Although it has been reported that cholinesterase inhibitors can be used to slow the progression of dementia, therapeutic effects are still quite limited, and there are many undesirable side effects. Therefore, there is currently no effective clinical method for curing dementia.

Therefore, searching for drugs from natural sources that can effectively treat dementia is the goal of relevant researchers in this field.

### CONTENT OF INVENTION

### Summary of Invention

Through research, the applicant unexpectedly discovered that a Chinese medicinal composition containing Hedysari Radix, Scutellariae Radix, Asparagi Radix and Chuanxiong Rhizoma can not only effectively treat Alzheimer's disease (AD), but can also ameliorate cognitive function, spatial memory and promote neurogenesis. In addition, the Chinese medicinal composition can treat ischemia-reperfusion-induced nerve injury, and is therefore considered to have high potential for use in treating dementia.

Therefore, in a first aspect, the present invention provides a Chinese medicinal composition that includes: Hedysari Radix, Scutellariae Radix, Asparagi Radix, and Chuanxiong Rhizoma.

In a second aspect, the present invention provides use of a Chinese medicinal composition in the manufacture of a medicament for treating dementia, wherein the Chinese medicinal composition includes: Hedysari Radix, Scutellariae Radix, Asparagi Radix, and Chuanxiong Rhizoma.

In a third aspect, the present invention provides a method for treating dementia, which includes administering to a subject in need thereof a Chinese medicinal composition, wherein the Chinese medicinal composition includes: Hedysari Radix, Scutellariae Radix, Asparagi Radix, and Chuanxiong Rhizoma.

### BRIEF DESCRIPTION OF DRAWINGS

The above and other objects, features and advantages of the present invention will become apparent with reference to the following detailed description and preferred embodiments and accompanying drawings, in which:

FIG. 1 shows the discrimination index measured for each group of mice in Example 2, where “*”, “**” and “***” respectively represent p<0.05, p<0.01 and p<0.001;

FIG. 2 shows an elution profile obtained from subjecting a Chinese medicinal composition 3 of the present invention to high performance liquid chromatography;

FIG. 3 shows the weight burrowed measured for each group of mice in Example 4, where “*” and “**” respectively represent p<0.05 and p<0.01;

FIG. 4 shows the nest score measured for each group of mice in Example 4, where “***” represents p<0.001;

FIG. 5 shows the escape latency measured for each group of mice in Example 4, where “*” represents p<0.05;

FIG. 6 shows the number of Aβ plaques measured in the brains of each group of mice in Example 4, where “***” represents p<0.001;

FIG. 7 shows the soluble Aβ40 content measured in the cerebral cortexes of each group of mice in Example 4, where “*” and “***” respectively represent p<0.05 and p<0.001;

FIG. 8 shows the escape latency measured for each group of mice in Example 5, where “*” and “**” respectively represent p<0.05 and p<0.01;

FIG. 9 shows the number of Aβ plaques measured in the brains of each group of mice in Example 5, where “*” and “***” respectively represent p<0.05 and p<0.001;

FIG. 10 shows the number of DCX⁺ cells measured in the subgranular zone of the dentate gyrus of the brains of each group of mice in Example 5, where “*” and “**” respectively represent p<0.05 and p<0.01; and

FIG. 11 shows results of immunofluorescence staining assay of the hippocampus of the brains of each group of mice in Example 7.

### DETAILED DESCRIPTION

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present invention belongs. One skilled in the art will recognize many methods and materials similar or equivalent to those described herein, which could be used in the practice of the present invention. Of course, the present invention is in no way limited to the methods and materials described.

The present invention provides a Chinese medicinal composition that includes: Hedysari Radix, Scutellariae Radix, Asparagi Radix, and Chuanxiong Rhizoma. In certain embodiments, the Chinese medicinal composition includes: 7 to 11 parts by weight of Hedysari Radix, 2 to 6 parts by weight of Scutellariae Radix, 4 to 7 parts by weight of Asparagi Radix, and 2 to 4 parts by weight of Chuanxiong Rhizoma. In an exemplary embodiment, the Chinese medicinal composition includes: 8 to 10 parts by weight of Hedysari Radix, 3 to 5 parts by weight of Scutellariae Radix, 5 to 6 parts by weight of Asparagi Radix, and 3 parts by weight of Chuanxiong Rhizoma.

According to the present invention, the Chinese medicinal composition may further include Cervi Cornu Pantotrichum. In certain embodiments, the Chinese medicinal composition includes: 7 to 11 parts by weight of Hedysari Radix, 2 to 6 parts by weight of Scutellariae Radix, 4 to 7 parts by weight of Asparagi Radix, 2 to 4 parts by weight of Chuanxiong Rhizoma, and 1 to 3 parts by weight of Cervi Cornu Pantotrichum. In an exemplary embodiment, the Chinese medicinal composition includes: 8 to 10 parts by weight of Hedysari Radix, 3 to 5 parts by weight of Scutellariae Radix, 5 to 6 parts by weight of Asparagi Radix, 3 parts by weight of Chuanxiong Rhizoma, and 2 parts by weight of Cervi Cornu Pantotrichum.

According to the present invention, in additional to containing the above-mentioned five types of ingredients, the Chinese medicinal composition may further include Aconiti Lateralis Radix Preparata. In certain embodiments, the Chinese medicinal composition includes: 7 to 11 parts by weight of Hedysari Radix, 2 to 6 parts by weight of Scutellariae Radix, 4 to 7 parts by weight of Asparagi Radix, 2 to 4 parts by weight of Chuanxiong Rhizoma, 1 to 3 parts by weight of Cervi Cornu Pantotrichum, and 1 to 3 parts by weight of Aconiti Lateralis Radix Praeparata. In an exemplary embodiment, the Chinese medicinal composition includes: 8 to 10 parts by weight of Hedysari Radix, 3 to 5 parts by weight of Scutellariae Radix, 5 to 6 parts by weight of Asparagi Radix, 3 parts by weight of Chuanxiong Rhizoma, 2 parts by weight of Cervi Cornu Pantotrichum, and 2 parts by weight of Aconiti Lateralis Radix Praeparata.

According to the present invention, the Hedysari Radix, the Scutellariae Radix, the Asparagi Radix, the Chuanxiong Rhizoma, the Cervi Cornu Pantotrichum, and the Aconiti Lateralis Radix Praeparata in the Chinese medicinal composition may be fresh traditional Chinese medicinal materials, or may be products obtained through a process selected from the group consisting of the following: a drying treatment, a grinding treatment, a chopping treatment, a comminuting treatment, a processing treatment, and combinations thereof. In certain embodiments, the Aconiti Lateralis Radix Praeparata in the Chinese medicinal composition is subjected to a processing treatment to remove toxicity, and is also called Zhi Fu Zi or Paw Fuh Tzyy.

According to the present invention, the Hedysari Radix in the Chinese medicinal composition may come from dried roots of Hedysarum polybotrys Hand.-Mazz. The Scutellariae Radix in the Chinese medicinal composition may come from dried roots of Scutellaria baicalensis Georgi. The Asparagi Radix in the Chinese medicinal composition may come from dried roots of Asparagus cochinchinensis (Lour.) Merr. The Chuanxiong Rhizoma in the Chinese medicinal composition may come from dried rhizomes of Ligusticum chuanxiong Hort. The Cervi Cornu Pantotrichum in the Chinese medicinal composition may come from dried young antlers of species of the genus Alces, Axis, Cervus, Elaphurus, Rangifer or Rusa of the Cervidae family, especially from dried young antlers of Cervus elaphus Linnaeus, Cervus nippon taiouanus, or Rusa unicolor swinhoii. In an exemplary embodiment, the Cervi Cornu Pantotrichum comes from dried young antlers of Cervus elaphus Linnaeus. The Aconiti Lateralis Radix Praeparata in the Chinese medicinal composition may come from dried roots of Aconitum carmichaelii Debeaux.

According to the present invention, the Chinese medicinal composition may be manufactured into a dosage form suitable for oral administration using techniques well known to those skilled in the art. According to the present invention, the dosage form suitable for oral administration includes, but is not limited to: commercially available traditional Chinese medicine powder, or traditional Chinese medicine liquid or traditional Chinese medicine extract obtained by decocting traditional Chinese medicinal materials, as well as capsules and other preparations further made from the above-mentioned traditional Chinese medicine powder, traditional Chinese medicine liquid or traditional Chinese medicine extract. In certain embodiments, the Chinese medicinal composition is made by mixing traditional Chinese medicine powders or traditional Chinese medicine extracts of Hedysari Radix, Scutellariae Radix, Asparagi Radix, Chuanxiong Rhizoma, Cervi Cornu Pantotrichum and Aconiti Lateralis Radix Praeparata.

According to the present invention, the traditional Chinese medicinal materials (i.e., Hedysari Radix, Scutellariae Radix, Asparagi Radix, Chuanxiong Rhizoma, Cervi Cornu Pantotrichum and Aconiti Lateralis Radix Praeparata) used to prepare the Chinese medicinal composition may be decocted together or individually. In certain embodiments, the Chinese medicinal composition is obtained by mixing the traditional Chinese medicinal materials and then decocting them in water. According to the present invention, the weight ratio of the traditional Chinese medicinal materials to water may fall within the range of 1:8 to 1:25. In certain embodiments, the weight ratio of the traditional Chinese medicinal materials to water falls within the range of 1:10 to 1:12. In an exemplary embodiment, the weight ratio of the traditional Chinese medicinal materials to water is 1:10. According to the present invention, the decocting temperature may fall within the range of 70 to 100° C. In an exemplary embodiment, the decocting temperature is 100° C. According to the present invention, the decocting duration may fall within the range of 30 to 120 minutes. In certain embodiments, the decocting duration falls within the range of 30 to 90 minutes. In an exemplary embodiment, the decocting duration is 60 minutes.

The present invention also provides use of the abovementioned Chinese medicinal composition in the manufacture of a medicament for treating dementia.

As used herein, “treating” or “treatment” means preventing, reducing, alleviating, ameliorating, relieving, or controlling one or more clinical signs of a disease or disorder, as well as lowering, stopping or reversing the progression of the severity of a condition or symptom being treated.

According to the present invention, dementia treatment includes promoting neurogenesis. As used herein, the terms “neurogenesis” and “neuroregeneration” can be used interchangeably.

According to the present invention, the dementia is selected from the group consisting of: degenerative dementia, vascular dementia, and mixed dementia. In certain embodiments, the dementia is a degenerative dementia selected from the group consisting of: Alzheimer's disease (AD), dementia with Lewy body (DLB), Frontal-temporal dementia (FTD), Parkinson's disease dementia (PDD) and Huntington's disease (HD). In an exemplary embodiment, the dementia is Alzheimer's disease.

According to the present invention, the medicament may further include a pharmaceutically acceptable carrier that is widely employed in the art of drug-manufacturing. For example, the pharmaceutically acceptable carrier may include one or more agents selected from the following: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, taste making agents, absorption enhancers, sustained-release agents, molding agents, adhesive agents, nutritional additives, colorants, and the like. The choice and amount of these agents are within the expertise and routine skills of those skilled in the art.

According to the present invention, the medicament may be manufactured, using techniques well known to those skilled in the art, into a dosage form suitable for oral administration, including, but not limited to: sterile powders, tablets, troches, lozenges, pellets, pills, capsules, powders, dispersible powders or granules, solutions, suspensions, emulsions, syrups, elixirs, slurries, pastes for internal use, and the like.

The present invention also provides a method for treating dementia, which includes administering to a subject in need thereof the abovementioned Chinese medicinal composition.

As used herein, the terms “administering” and “administration” can be used interchangeably, and mean introducing, providing or delivering a predetermined active ingredient to a subject through any suitable routes to perform its intended function.

As used herein, the term “subject” means any mammal of interest, such as humans, monkeys, cows, sheep, horses, pigs, goats, dogs, cats, mice and rats.

According to the present invention, the dosage and frequency of administration of the medicament may vary depending on the following factors: the severity of the disease to be treated, routes of administration, and age, physical condition and response of the subject to be treated. In general, the medicament may be in the form of a single dose or divided into several doses.

### DETAILED DESCRIPTION OF PREFERRED EMBODIMENTS

The present invention will be further described by way of the following examples. However, it should be understood that these examples are only for illustration and should not be construed as limiting the present invention in practice.

### EXAMPLES

### General Experimental Materials

### 1. Experimental Animals:

Male APP/PS1 mice (4-5 months old, weighing about 30 g) and male C57/BL6 mice (4-5 months old, weighing about 25 g) used in the following examples were purchase from the National Laboratory Animal Center, R.O.C., and male ICR mice (adult, weighing about 28-30 g) used in the following examples were purchased from BioLasco Taiwan Co., Ltd. All the experimental animals were individually housed in a breeding box with a light and dark period of 12 hours each, a room temperature maintained at 22±1° C., and a relative humidity maintained at 65±5%, and water and feed were adequately supplied.

### General Experimental Method

### 1. Statistical Analysis:

In the following examples, experiments of each group were repeated 3 times, and the experimental data were expressed as “mean±standard deviation (SD)”, and analyzed by one-way analysis of variance (ANOVA) followed by Tukey's hones significance difference tests as a post hoc test, so as to evaluate the differences between the groups. If the statistical analysis result is p<0.05, it indicates statistical significance.

### Example 1. Preparation of Chinese Medicinal Composition of the Present Invention

The traditional Chinese medicinal materials were mixed according to Table 1 below, and the resulting mixture was then soaked in 1000 ml of hot water for 30 minutes. Next, the mixture was boiled at 100° C. for about 60 minutes until about 300 ml of liquid remained, and then filtered with filter paper (No. 1) to collect the filtrate. Afterwards, the collected filtrate was subjected to freeze-drying to obtain Chinese medicinal compositions 1 to 3 of the present invention.

TABLE 1
Formulas of Chinese Medicinal Compositions
1 to 3 of the Present Invention

| | Chinese Medicinal Composition | | | | | |
|---|---|---|---|---|---|---|
| | 1 | | 2 | | 3 | |
| | Amount | Parts by | Amount | Parts by | Amount | Parts by |
| Ingredient | (g) | Weight | (g) | Weight | (g) | Weight |
| Hedysari Radix | 37.5 | 10 | 37.5 | 10 | 37.5 | 10 |
| Scutellariae | 18.75 | 5 | 18.75 | 5 | 18.75 | 5 |
| Radix | | | | | | |
| Asparagi Radix | 18.75 | 5 | 18.75 | 5 | 18.75 | 5 |
| Chuanxiong | 11.25 | 3 | 11.25 | 3 | 11.25 | 3 |
| Rhizoma | | | | | | |
| Cervi Cornu | 0 | 0 | 7.5 | 2 | 7.5 | 2 |
| Pantotrichum | | | | | | |
| Aconiti | 0 | 0 | 0 | 0 | 7.5 | 2 |
| Lateralis Radix | | | | | | |
| Praeparata | | | | | | |

### Example 2. Effect of Chinese Medicinal Composition of the Present Invention on Recognition Memory Function of Mice with Alzheimer's Disease (AD)

### Experimental Method

### A. Administration of Chinese Medicinal Composition of the Present Invention:

First, APP/PS1 mice (5 months old) were randomly divided into one pathological control group and three experimental groups (i.e., experimental groups 1 to 3), and C57/BL6 mice (5 months old) served as a normal control group, n=5 in each group. The mice in the experimental groups 1 to 3 were respectively administered, via oral gavage, with the Chinese medicinal compositions 1 to 3 prepared in Example 1 (all at a dose of 100 mg/kg, all in normal saline), while the mice in the pathological control group and the mice in the normal control group were administered, via oral gavage, with an equal amount of normal saline, once a day for a total of 5 weeks. At the end of the 5^{th} week after the start of drug administration, the mice in each group were subjected to the test in item B below.

### B. Novel Object Recognition Test:

This test was conducted for a total of 3 days in an open box that had been cleaned with ethanol to remove odors. On the first day, the mice in each group were placed in the center of the open box for 5 to 10 minutes to allow the mice to acclimate to the environment. On the second day, the mice in each group were placed in the center of the open box, and two identical objects were placed in two diagonal corners for the mice to explore for 10 minutes. On the third day, the mice in each group were placed in the center of the open box, and one of the two objects was replaced with a different object (i.e., a new object and an old object were respectively placed in the two diagonal corners) for the mice to explore for 5 minutes, and the time the mice spent exploring the new object and the old object was recorded. “Object exploration” is defined as a mouse approaching an object and making physical contact with the object with its snout and/or forepaw.

The discrimination index was calculated by substituting the measured exploration time of the new object and the old object into the following Equation (1):

$$A = B / ( B + C ) \qquad Equation ( 1 )$$

$$Where :$$

$$A = discrimination index$$

$$B = exploration time of new object$$

$$C = exploration time of old object$$

Afterwards, the obtained experimental data were analyzed according to the method described in item 1 of “General Experimental Method” above.

### Results

### A. Novel Object Recognition Test:

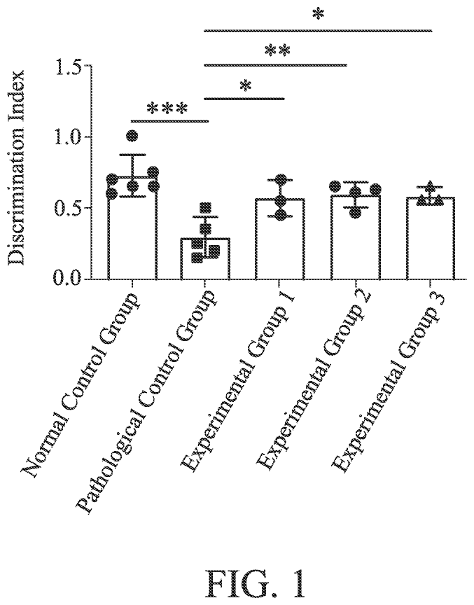

FIG. 1 shows the discrimination index measured for each group of mice. As shown in FIG. 1, compared with the normal control group, the discrimination index of the mice in the pathological control group showed a significant decrease. This indicates that the APP/PS1 mice had developed AD, which had caused defects in their ability to recognize new objects. In addition, compared with the pathological control group, the discrimination indexes of the mice in experimental groups 1 to 3 showed significant increases. These experimental results show that the Chinese medicinal compositions 1 to 3 of the present invention can ameliorate recognition and memory functions of mice with AD and have a high potential for treating AD.

Next, the applicant selected Chinese medicinal composition 3 as a representative to carry out the following experiment.

### Example 3. High Performance Liquid Chromatography (HPLC) Analysis of Chinese Medicinal Composition of the Present Invention

### Experimental Method

The Chinese medicinal composition 3 of the present invention was dissolved in ddH₂O to obtain a mixture with a final concentration of 50 mg/ml, and then the mixture was filtered using a filter membrane with a pore size of 0.22 μm. Next, the filtrate was collected to serve as a test sample, and the test sample was subjected to HPLC analysis. The HPLC analysis instrument used was as follows: SHIMADZU LC-2050C HPLC system; the analysis column was COSMOSIL® C18-AR-II ODS column, length: 250 mm×4.6 mm. The operating conditions for HPLC are shown in Table 2 below.

TABLE 2
HPLC Operating Conditions

| Operating | |
|---|---|
| Parameters | Conditions |
| Detection | 210 nm, 254 nm and 280 nm |
| Wavelength | |
| Mobile Phase | Acetonitrile containing 0.3 vol % |
| | phosphoric acid/0.3 vol % |
| | phosphoric acid, 100:0 (v/v) |
| Gradient | Acetonitrile was maintained at |
| Elution of | 100% from the 0^{th} to the 3^{rd} |
| Mobile Phase | minute; acetonitrile changed from |
| | 100% to 97% from the 3^{rd} to |
| | the 10^{th} minute; acetonitrile was |
| | maintained at 97% from the |
| | 10^{th} to the 13^{th} minute; acetonitrile |
| | changed from 97% to 90% |
| | from the 13^{th} to the 18^{th} minute; |
| | acetonitrile changed from |
| | 90% to 85% from the 18^{th} to |
| | the 35^{th} minute; acetonitrile |
| | changed from 85% to 75% from |
| | the 35^{th} to the 50^{th} minute; |
| | acetonitrile changed from 75% to |
| | 70% from the 50^{th} to the 65^{th} |
| | minute; acetonitrile changed |
| | from 70% to 0% from the 65^{th} to |
| | the 75^{th} minute; acetonitrile |
| | was maintained at 0% from the |
| | 75^{th} to the 85^{th} minute. |
| Flow Rate | 1 |
| (mL/min) | |

### Results

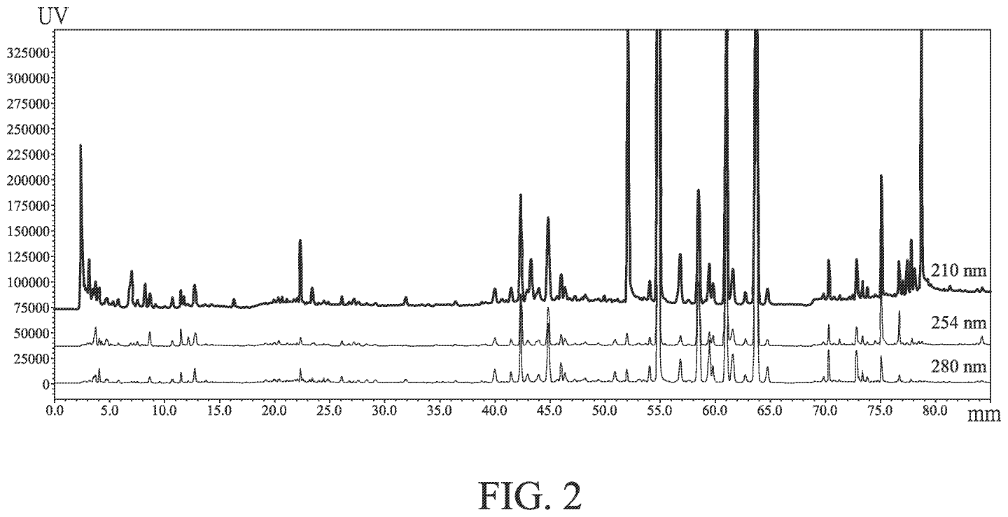

FIG. 2 shows an elution profile obtained by subjecting the Chinese medicinal composition 3 of the present invention to HPLC. As shown in FIG. 2, the Chinese medicinal composition 3 of the present invention had many peaks during the retention period from the 0^{th} to the 85^{th} minute, indicating that the composition is quite complex.

### Example 4. Evaluation of Therapeutic Effect of Chinese Medicinal Composition of the Present Invention on Mice with AD

### Experimental Method

### A. Administration of Chinese Medicinal Composition of the Present Invention:

First, APP/PS1 mice (5 months old) were randomly divided into one pathological control group and one experimental group, and C57/BL6 mice (5 months old) served as a normal control group, n=5 in each group. The mice in the experimental group were administered, via oral gavage, with the Chinese medicinal composition 3 prepared in Example 1 (at a dose of 100 mg/kg, in normal saline), while the mice in the pathological control group and the mice in the normal control group were administered, via oral gavage, with an equal amount of normal saline, once a day for a total of 8 weeks. At the end of the 5^{th} week after the start of drug administration, the mice in each group were subjected to the tests in items B and C below. At the end of the 6^{th} week after the start of drug administration, the mice in each group were subjected to the test in item D below. At the end of the 8^{th} week after the start of drug administration, the mice in each group were sacrificed and their brains were removed and then subjected to the analyses of items E and F below.

### B. Burrowing Test:

A cylinder containing 230 g of food pellet was placed in a cage for each group of mice to dig, and then the remaining food pellets in the cylinder were weighed after 2 hours.

Afterwards, the obtained experimental data were analyzed according to the method described in item 1 of “General Experimental Method” above.

### C. Nesting Test:

Two cotton pieces were placed in the cage as nesting materials, and then after 24 hours, the mice in each group were observed to see whether they tore up the nesting materials and used them to build nests. The nests were scored with a score of 1 to 5 based on their completeness, with higher values indicating higher degrees of nest completeness.

Afterwards, the obtained experimental data were analyzed according to the method described in item 1 of “General Experimental Method” above.

### D. Morris Water Maze Test:

A water maze was set in a circular pool (120 cm in diameter and 40cm in depth) with a water level of 20 cm and a water temperature maintained at 22-24° C. The pool was divided into four equal quadrants, and a platform (10 cm in diameter) was placed 1 cm below the water surface in one quadrant.

First, the mice in each group were subjected to a 6-day training session, with four trials per day and 20 minutes between each trial. In each trial, the mice in each group were randomly placed in one of three starting positions in the pool and faced the pool wall. Regardless of whether a mouse could find the platform within 60 seconds, the mouse was allowed to remain on the platform for 30 seconds. During the trials on the 6^{th} day, a computer video image analysis system was used to record as the escape latency the average time taken by the mice to find the platform in the four trials.

Afterwards, the obtained experimental data were analyzed according to the method described in item 1 of “General Experimental Method” above.

### E. Immunofluorescence Staining Assay:

Brains of the mice in each group were fixed with 4% formaldehyde at 4° C. and then sliced (thickness of about 30 μm) using a freezing microtome. The obtained tissue sections were analyzed by immunofluorescence staining assay using anti-Aβ1-16 antibody (AB10, Millipore cat#MAB5208) as a primary antibody and Alexa-Fluor®647-conjugated anti-IgG antibody (Jackson ImmunoResearch) as a secondary antibody and according to techniques well known and commonly used by those skilled in the art. The stained tissue sections were observed and photographed using a confocal fluorescence microscope (Zeiss LSM 780), and the numbers of Aβ plaques were calculated using Image J software.

Afterwards, the obtained experimental data were analyzed according to the method described in item 1 of “General Experimental Method” above.

### F. Enzyme-Linked Immunosorbent Assay (ELISA):

Cerebral cortexes of the mice in each group were placed in a homogenization buffer containing 4% SDS and protease inhibitors to form a mixture, and then the mixture was subjected to ultrasonic treatment. After centrifugation at 100,000×g for one hour at 4° C., the supernatant was collected and neutralized with Tris buffer (1M, pH 11) to obtain a test sample. Then, the content of soluble Aβ40 in the test sample was measured using an Amyloid beta 40 ELISA kit (Invitrogen, KHB3482) according to the manufacturer's instructions.

Afterwards, the obtained experimental data were analyzed according to the method described in item 1 of “General Experimental Method” above.

### Results

### A. Burrowing Test:

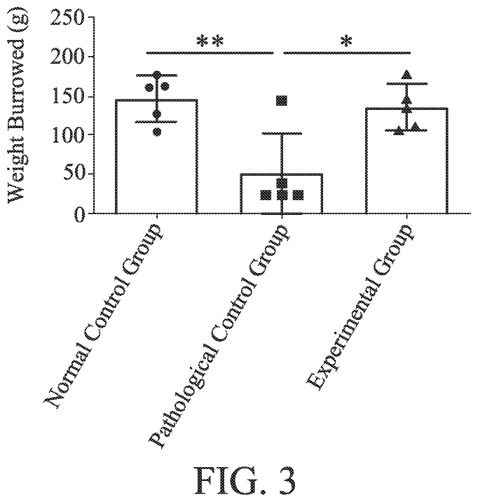

FIG. 3 shows the weight burrowed measured for each group of mice. As shown in FIG. 3, compared with the normal control group, the weight burrowed of the mice in the pathological control group showed a significant decrease. This indicates that the APP/PS1 mice had developed AD, which had caused defects in their innate behavior ability. In addition, compared with the pathological control group, the weight burrowed of the mice in the experimental group showed a significant increase, and was even close to that of the mice in the normal control group. These experimental results show that the Chinese medicinal composition of the present invention can effectively treat AD and ameliorate the innate behavior ability of mice with AD.

### B. Nesting Test:

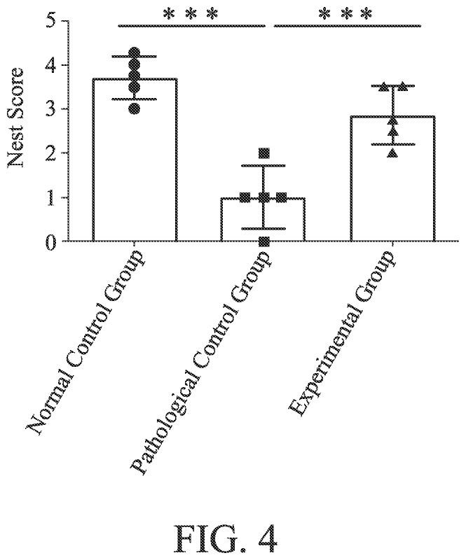

FIG. 4 shows the nest score measured for the mice in each group. As shown in FIG. 4, compared with the normal control group, the nest score of the mice in the pathological control group showed a significant decrease. This indicates that the APP/PS1 mice had developed AD, which had caused defects in their activities of daily living (ADLs). In addition, compared with the pathological control group, the nest score of the mice in the experimental group showed a significant increase, and was even close to that of the mice in the normal control group. These experimental results show that the Chinese medicinal composition of the present invention can effectively treat AD and improve the ability of mice with AD to perform ADLs.

### C. Morris Water Maze Test:

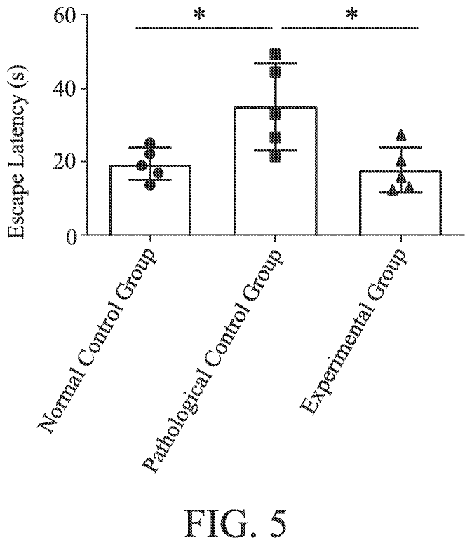

FIG. 5 shows the escape latency measured for the mice in each group. As shown in FIG. 5, compared with the normal control group, the escape latency of the mice in the pathological control group showed a significant increase. This indicates that the APP/PS1 mice had developed AD, which had caused defects in their spatial memory. In addition, compared with the pathological control group, the escape latency of the mice in the experimental group showed a significant decrease, and was even close to that of the mice in the normal control group. These experimental results show that the Chinese medicinal composition of the present invention can effectively treat AD and ameliorate the spatial memory of mice with AD.

### D. Immunofluorescence Staining Assay:

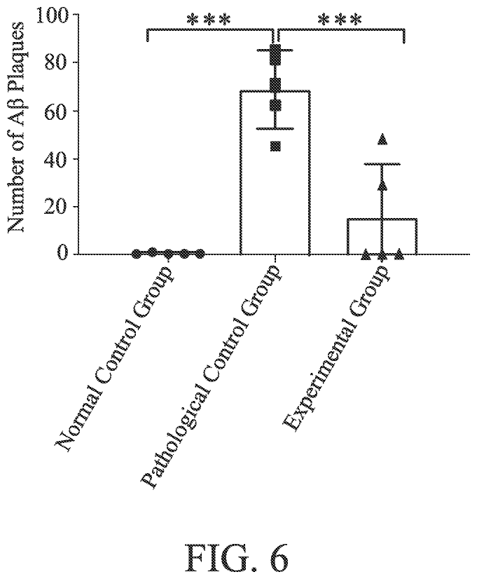

FIG. 6 shows the number of Aβ plaques measured in the brains of the mice in each group. As shown in FIG. 6, compared with the normal control group, the number of Aβ plaques in the mice in the pathological control group showed a significant increase. This indicates that the APP/PS1 mice had developed AD and abnormally accumulated a large number of Aβ plaques in their brains. In addition, compared with the pathological control group, the number of Aβ plaques in the mice in the experimental group showed a significant decrease. These experimental results show that the Chinese medicinal composition of the present invention can effectively treat AD and ameliorate Aβ plaque accumulation.

### E. Enzyme-Linked Immunosorbent Assay (ELISA):

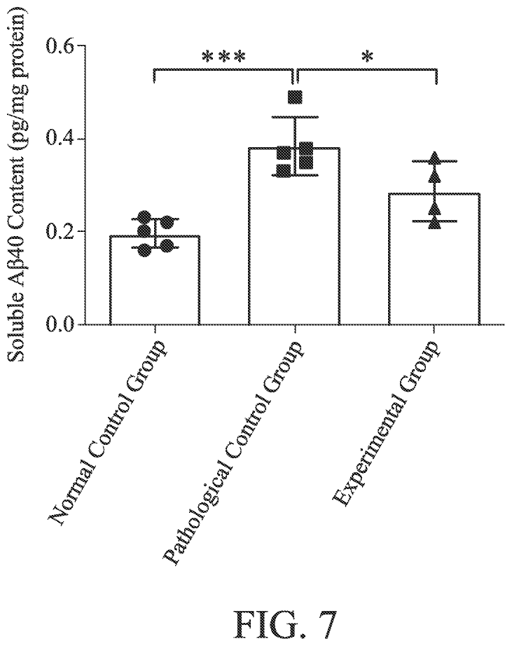

FIG. 7 shows the soluble Aβ40 content measured in the cerebral cortexes of the mice in each group. As shown in FIG. 7, compared with the normal control group, the soluble Aβ40 content in the mice in the pathological control group showed a significant increase. This indicates that the APP/PS1 mice had developed AD and produced a large amount of soluble Aβ40 in their brains. In addition, compared with the pathological control group, the soluble Aβ40 content in mice in the experimental group showed a significant decrease. These experimental results show that the Chinese medicinal composition of the present invention can effectively treat AD and reduce the generation of soluble Aβ40.

### Example 5. Effect of Dosage of Chinese Medicinal Composition of the Present Invention on Therapeutic Efficacy on Mice with AD

### Experimental Method

### A. Administration of Chinese Medicinal Composition of the Present Invention:

First, APP/PS1 mice (4 months old) were randomly divided into one pathological control group and two experimental groups (i.e., experimental groups 1 and 2), and C57/BL6 mice (4 months old) served as a normal control group, n=5 in each group. The mice in the experimental group 1 and the mice in the experimental group 2 were administered, via oral gavage, with the Chinese medicinal composition 3 prepared in Example 1 (at doses of 50 and 300 mg/kg, respectively, all in normal saline), while the mice in the pathological control group and the mice in the normal control group were administered, via oral gavage, with an equal amount of normal saline, once a day for a total of 18 weeks. At the end of the 14^{th} week after the start of drug administration, the mice in each group were subjected to the test in item B below. At the end of the 18th week after the start of drug administration, the mice in each group were sacrificed and their brains were removed and then subjected to the analysis of item C below.

### B. Morris Water Maze Test:

This test was carried out according to the method described in item D of Example 4 above.

### C. Immunofluorescence Staining Assay:

First, brain tissue sections of the mice in each group were prepared according to the method described in item E of Example 4 above. Then, some of the obtained brain tissue sections were selected, and analyzed by immunofluorescence staining assay and the numbers of Aβ plaques were calculated according to the method described in item E of Example 4 above.

In addition, hippocampal tissue sections were selected from the obtained brain tissue sections, and then immunofluorescence staining assay was performed by using anti-doublecortin (DCX) antibody (Abcam, Cat#ab18723) as a primary antibody and Alexa-Fluor®647-conjugated anti-IgG antibody (Jackson ImmunoResearch) as a secondary antibody and according to techniques well known and commonly used by those skilled in the art. The stained tissue sections were observe and photographed using a confocal fluorescence microscope, and then the numbers of DCX⁺ cells in the subgranular zone of the dentate gyruses were calculated using Image J software.

Afterwards, the obtained experimental data were analyzed according to the method described in item 1 of “General Experimental Method” above.

### Results

### A. Morris Water Maze Test:

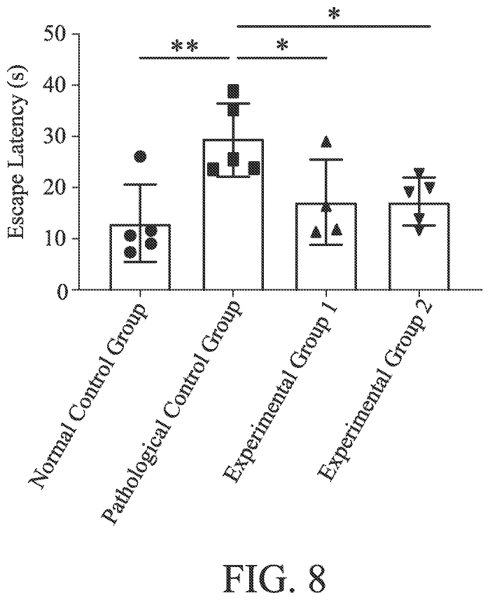

FIG. 8 shows the escape latency measured for each group of mice. As shown in FIG. 8, compared with the normal control group, the escape latency of the pathological control group showed a significant increase. This indicates that the APP/PS1 mice had developed AD, which had caused defects in their spatial memory. In addition, compared with the pathological control group, the escape latencies of the mice in experimental groups 1 and 2 showed significant decreases, and were even close to that of the mice in the normal control group. These experimental results show that the Chinese medicinal composition of the present invention can effectively treat AD and ameliorate the spatial memory of mice with AD at different doses.

### B. Immunofluorescence Staining Assay:

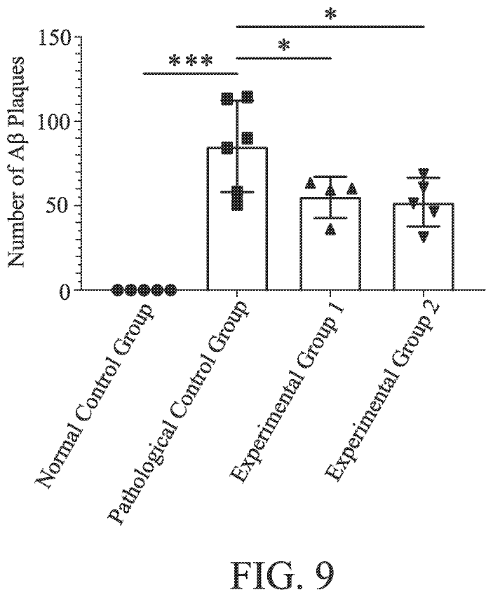

FIG. 9 shows the number of Aβ plaques measured in the brains of the mice in each group. As shown in FIG. 9, compared with the normal control group, the number of Aβ plaques in the mice in the pathological control group showed a significant increase. This indicates that the APP/PS1 mice had developed AD and abnormally accumulated a large number of Aβ plaques in their brains. In addition, compared with the pathological control group, the numbers of Aβ plaques in the mice in the experimental groups 1 and 2 showed significant decreases.

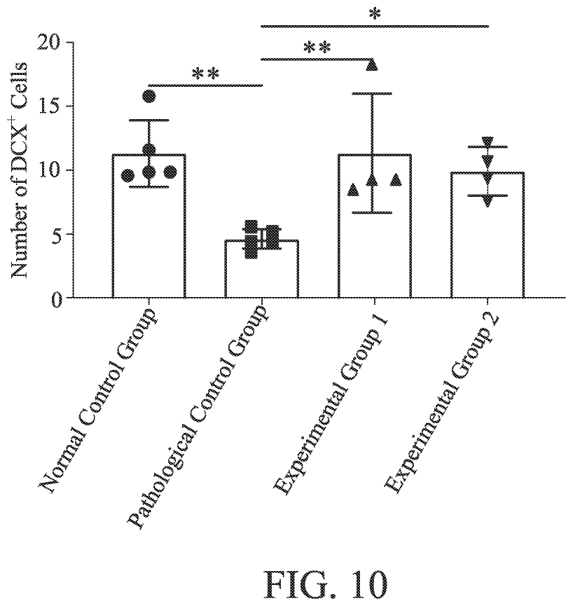

FIG. 10 shows the number of DCX⁺ cells measured in the subgranular zone of the dentate gyrus of the brains of the mice in each group. As shown in FIG. 10, compared with the normal control group, the number of DCX⁺ cells in the mice in the pathological control group showed a significant decrease. This indicates that the APP/PS1 mice had developed AD and inhibited neurogenesis in the hippocampus of their brains. In addition, compared with the pathological control group, the numbers of DCX⁺ cells in the mice in the experimental groups 1 and 2 showed significant increases.

These experimental results show that the Chinese medicinal composition of the present invention can ameliorate Aβ plaque accumulation and promote neurogenesis at different doses, thereby achieving the effect of treating AD.

### Example 6. Evaluation of Efficacy of Chinese Medicinal Composition of the Present Invention in Ameliorating AD Patients

### A. Preparation of Chinese Medicinal Composition 4 of the Present Invention:

Six ingredients in powder form were mixed according to the following Table 3 to obtain Chinese medicinal composition 4 of the present invention.

TABLE 3
Formula of Chinese Medicinal Composition 4 of the Present Invention

| | | Parts by |
|---|---|---|
| Ingredient | Amount (g) | Weight |
| Hedysari Radix | 4.0 | 8 |
| Scutellariae Radix | 1.5 | 3 |
| Asparagi Radix | 3.0 | 6 |
| Chuanxiong Rhizoma | 1.5 | 3 |
| Cervi Cornu Pantotrichum | 1.0 | 2 |
| Aconiti Lateralis Radix Praeparata | 1.0 | 2 |

### B. Experimental Method and Results:

Test subjects who participated in this experiment were all from Tri-Service General Hospital and were diagnosed with mild to moderate AD through outpatient clinics. A total of 42 test subjects participated.

All test subjects were randomly divided into an experimental group (n=25) and a pathological control group (n=17), wherein the test subjects in the experimental group were orally administered with the Chinese medicinal composition 4 (dose of 6 g) prepared in item A of this example, twice a day for a total of 3 months. Before the start of and after the end of drug administration, the test subjects were scored using the mini-mental state examination (MMSE).

Results obtained are shown in Table 4 below.

TABLE 4
MMSE Scores of Test Subjects

| | Experimental Group | Pathological Control Group |
|---|---|---|
| Before Start of Drug | 21.36 ± 14.30 | 21.62 ± 3.90 |
| Administration | | |
| After End of Drug | 22.52 ± 3.58 | 21.35 ± 3.41 |
| Administration | | |
| Change Value | 1.16 ± 2.17* | −0.29 ± 2.17 |

* Compared with pathological control group, p < 0.05

As shown in Table 4, after 3 months of drug administration, MMSE scores of the test subjects in the experimental group increased significantly, while those of the pathological control group decreased slightly. These experimental results show that the Chinese medicinal composition of the present invention can effectively treat AD and ameliorate cognitive function.

### Example 7. Evaluation of Therapeutic Efficacy of Chinese Medicinal Composition of the Present Invention on Mice With Ischemia-Reperfusion-Induced Nerve Injury

### Experimental Method

### A. Induction of Nerve Injury and Administration of Chinese Medicinal Composition of the Present Invention:

First, ICR mice were randomly divided into a normal control group, a pathological control group, and an experimental group, n=5 in each group. Next, the mice in the pathological control group and the mice in the experimental group were anesthetized with a mixture of 1.5-2% isoflurane and oxygen, and then the midlines of the necks of the mice were incised to expose the common carotid artery, external carotid artery and internal carotid artery, followed by conducting the following middle cerebral artery occlusion/reperfusion (MCAO/R) operation procedures: a heat-blunted nylon monofilament surgical suture (about 100 μm in diameter) coated with silicone was placed into the exposed external carotid artery and advanced to the internal carotid artery and into the Willis' circle to occlude the right middle cerebral artery (MCA) and block blood flow. After 40 minutes, the surgical suture was removed to cause reperfusion of the MCA, thereby leaving the mice with ischemia-reperfusion-induced nerve injury. The mice in the normal control group received no treatment.

Starting at the second hour after MCAO/R, the mice in the experimental group were administered, via oral gavage, with the Chinese medicinal composition 3 prepared in Example 1 (at a dose of 1 g/kg, in normal saline), while the mice in the pathological control group and the mice in the normal control group were administered, via oral gavage, with an equal amount of normal saline, once a day until the 2^{nd} day after MCAO/R.

On days 0, 1 and 2 after MCAO/R, a survival number of the mice in each group was observed. The survival rate (%) of each group of mice was calculated by substituting the survival number thus obtained into the following

Equation (2):

$$D = ( E / 5 ) \times 100 \% \qquad Equation ( 2 )$$

$$Where :$$

$$D = survival rate ( \% )$$

$$E = surival number of each group$$

In addition, on the 1^{st} day after MCAO/R, the mice in the pathological control group and the mice in the experimental group were subjected to the analysis of item B below, and on the 2^{nd} day after MCAO/R and after analysis of the survival rates was completed, the mice in each group were sacrificed and their brains were removed and then subjected to the analysis of item C below.

### B. Analysis of Mobility:

The mice in the pathological control group and the mice in the experimental group were placed in a behavior observation box (60×40×60 cm³), and the distance traveled and area traveled of each mouse within 3 minutes were analyzed using a video-tracking system (SMART v2.5.21, Panlab, Spain).

Afterwards, the obtained experimental data were analyzed according to the method described in item 1 of “General Experimental Method” above.

### C. Immunofluorescence Staining Assay:

Brains of the mice in each group were fixed with 4% formaldehyde at 4° C. and then sliced (thickness of about 20 μm) using a freezing microtome.

The obtained tissue sections were analyzed by immunofluorescence staining assay using primary antibodies and secondary antibodies shown in Table 5 below and according to techniques well known and commonly used by those skilled in the art, and then mounted with an anti-fluorescence attenuation mounting medium containing the cell nuclear stain DAPI. The stained tissue sections were observed and photographed using a confocal fluorescence microscope (Zeiss LSM 780), and the numbers of neuron stem cells in the hippocampus and the lateral ventricle were calculated using Zen 2011 imaging software (black edition, Carl Zeiss MicroImaging GmbH, 1997-2011).

Afterwards, the obtained experimental data were analyzed according to the method described in item 1 of “General Experimental Method” above.

TABLE 5
Primary Antibodies and Secondary Antibodies for Immunofluorescence
Staining Assay

| Protein | Primary Antibody | Secondary Antibody |
|---|---|---|
| DCX | Anti-DCX Antibody | Alexa-Fluor ®488-Conjugated |
| | (abcam) | Anti-IgG Antibody |
| | | (Cell Signaling Technology, Inc.) |
| Iba-1 | Anti-Iba-1 Antibody | Alexa-Fluor ®647- Conjugated |
| | (IREAL) | Anti-IgG Antibody |
| | | (Cell Signaling Technology, Inc.) |
| CD206 | Anti-CD206 Antibody | Alexa-Fluor ®555-Conjugated |
| | (IREAL) | Anti-IgG Antibody |
| | | (Cell Signaling Technology, Inc.) |

### Results

### A. Analysis of Survival Rate:

Table 6 below shows the survival rate of each group of mice. As shown in Table 6, compared with the normal control group, the survival rate of the mice in the pathological control group decreased rapidly and significantly. This indicates that the MCAO/R successfully caused ischemia-reperfusion-induced nerve injury in the mice. In addition, compared with the pathological control group, the survival rate of the mice in the experimental group showed a significant increase.

TABLE 6
Survival Rate of Each Group of Mice (%)

| | Experimental | Pathological | Normal Control |
|---|---|---|---|
| | Group | Control Group | Group |
| Day 0 | 100 | 100 | 100 |
| Day 1 | 50 | 30 | 100 |
| Day 2 | 40 | 20 | 100 |

### B. Analysis of Mobility:

Table 7 below shows the distance traveled and the area traveled measured for each group of mice. As shown in Table 7, the distance traveled and the area traveled of the mice in the experimental group were significantly greater than those of the pathological control group.

TABLE 7
Distance Traveled and Area Traveled Measured for Each Group of Mice

| | Experimental Group | Pathological Control Group |
|---|---|---|
| Distance Traveled (m) | 14.73 ± 0.9* | 3.60 ± 0.7 |
| Area Traveled (%) | 81 ± 7* | 45 ± 6 |

*Compared with pathological control group, p < 0.05

### C. Immunofluorescence Staining Assay:

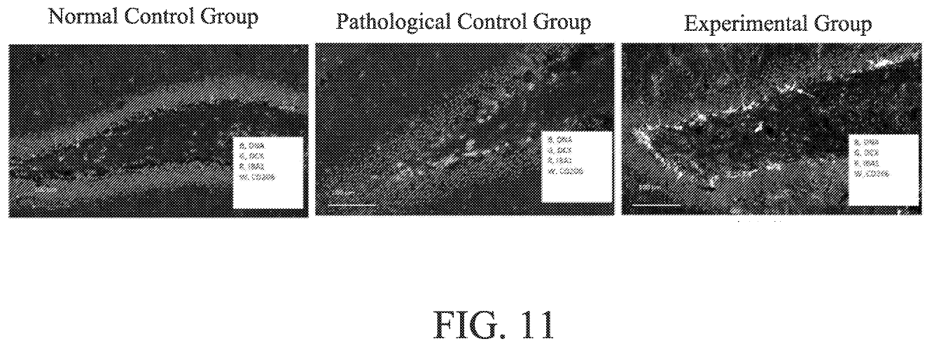

FIG. 11 shows results of immunofluorescence staining assay of the hippocampus of the brains of each group of mice. Table 8 below shows the numbers of neuron stem cells measured in the hippocampus and the lateral ventricle of the brains of each group of mice.

### Table 8. Number of Neuron Stem Cells Measured in Hippocampus and Lateral

Ventricle of Brains of Each Group of Mice

| | Experimental | Pathological | Normal |
|---|---|---|---|
| | Group | Control Group | Control Group |
| Hippocampus | 15 ± 5* | 2 ± 1 | 25 ± 5* |
| Lateral | 180 ± 15* | 81 ± 7 | 207 ± 12* |
| Ventricle | | | |

*Compared with pathological control group, p < 0.05

As shown in FIG. 11 and Table 8, compared with the normal control group, the number of neuron stem cells and microglia in the mice in the pathological control group showed a significant decrease. This indicates that the MCAO/R successfully caused ischemia-reperfusion-induced nerve injury in the mice. In addition, compared with the pathological control group, the mice in the experimental group had a significant proliferation of neuron stem cells and microglia. In particular, the number of neuron stem cells in the lateral ventricle was even close to that of the normal control group.

These experimental results show that the Chinese medicinal composition of the present invention can effectively treat ischemia-reperfusion-induced nerve injury and promote neurogenesis to repair damaged brain areas, thereby improving mobility and survival ability.

In summary of the above experimental results, the applicant believes that the Chinese medicinal composition of the present invention can be used in treating dementia, including degenerative dementia, vascular dementia and mixed dementia, especially AD.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the present invention. It should be further appreciated that in the description, various features are sometimes grouped together in a single embodiment, figure, or description thereof for the purpose of streamlining the present invention and aiding in the understanding of various inventive aspects. One or more features or specific details from one embodiment may be practiced together with one or more features or specific details from another embodiment, where appropriate, in the practice of the present invention.

While the present invention has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

1. A Chinese medicinal composition comprising: Hedysari Radix, Scutellariae Radix, Asparagi Radix, and Chuanxiong Rhizoma.

2. The Chinese medicinal composition as claimed in claim 1, which includes: 7 to 11 parts by weight of Hedysari Radix, 2 to 6 parts by weight of Scutellariae Radix, 4 to 7 parts by weight of Asparagi Radix, and 2 to 4 parts by weight of Chuanxiong Rhizoma.

3. The Chinese medicinal composition as claimed in claim 2, which includes: 8 to 10 parts by weight of Hedysari Radix, 3 to 5 parts by weight of Scutellariae Radix, 5 to 6 parts by weight of Asparagi Radix, and 3 parts by weight of Chuanxiong Rhizoma.

4. The Chinese medicinal composition as claimed in claim 1, further comprising Cervi Cornu Pantotrichum.

5. The Chinese medicinal composition as claimed in claim 4, which includes: 7 to 11 parts by weight of Hedysari Radix, 2 to 6 parts by weight of Scutellariae Radix, 4 to 7 parts by weight of Asparagi Radix, 2 to 4 parts by weight of Chuanxiong Rhizoma, and 1 to 3 parts by weight of Cervi Cornu Pantotrichum.

6. The Chinese medicinal composition as claimed in claim 5, which includes: 8 to 10 parts by weight of Hedysari Radix, 3 to 5 parts by weight of Scutellariae Radix, 5 to 6 parts by weight of Asparagi Radix, 3 parts by weight of Chuanxiong Rhizoma, and 2 parts by weight of Cervi Cornu Pantotrichum.

7. The Chinese medicinal composition as claimed in claim 4, further comprising Aconiti Lateralis Radix Praeparata.

8. The Chinese medicinal composition as claimed in claim 7, which includes: 7 to 11 parts by weight of Hedysari Radix, 2 to 6 parts by weight of Scutellariae Radix, 4 to 7 parts by weight of Asparagi Radix, 2 to 4 parts by weight of Chuanxiong Rhizoma, 1 to 3 parts by weight of Cervi Cornu Pantotrichum, and 1 to 3 parts by weight of Aconiti Lateralis Radix Praeparata.

9. The Chinese medicinal composition as claimed in claim 8, which includes: 8 to 10 parts by weight of Hedysari Radix, 3 to 5 parts by weight of Scutellariae Radix, 5 to 6 parts by weight of Asparagi Radix, 3 parts by weight of Chuanxiong Rhizoma, 2 parts by weight of Cervi Cornu Pantotrichum, and 2 parts by weight of Aconiti Lateralis Radix Praeparata.

10. A method for treating dementia, comprising administering to a subject in need thereof a Chinese medicinal composition including Hedysari Radix, Scutellariae Radix, Asparagi Radix, and Chuanxiong Rhizoma.

11. The method as claimed in claim 10, wherein the Chinese medicinal composition includes: 7 to 11 parts by weight of Hedysari Radix, 2 to 6 parts by weight of Scutellariae Radix, 4 to 7 parts by weight of Asparagi Radix, and 2 to 4 parts by weight of Chuanxiong Rhizoma.

12. The method as claimed in claim 11, wherein the Chinese medicinal composition includes: 8 to 10 parts by weight of Hedysari Radix, 3 to 5 parts by weight of Scutellariae Radix, 5 to 6 parts by weight of Asparagi Radix, and 3 parts by weight of Chuanxiong Rhizoma.

13. The method as claimed in claim 10, wherein the Chinese medicinal composition further includes Cervi Cornu Pantotrichum.

14. The method as claimed in claim 13, wherein the Chinese medicinal composition includes: 7 to 11 parts by weight of Hedysari Radix, 2 to 6 parts by weight of Scutellariae Radix, 4 to 7 parts by weight of Asparagi Radix, 2 to 4 parts by weight of Chuanxiong Rhizoma, and 1 to 3 parts by weight of Cervi Cornu Pantotrichum.

15. The method as claimed in claim 14, wherein the Chinese medicinal composition includes: 8 to 10 parts by weight of Hedysari Radix, 3 to 5 parts by weight of Scutellariae Radix, 5 to 6 parts by weight of Asparagi Radix, 3 parts by weight of Chuanxiong Rhizoma, and 2 parts by weight of Cervi Cornu Pantotrichum.

16. The method as claimed in claim 13, wherein the Chinese medicinal composition further includes Aconiti Lateralis Radix Praeparata.

17. The method as claimed in claim 16, wherein the Chinese medicinal composition includes: 7 to 11 parts by weight of Hedysari Radix, 2 to 6 parts by weight of Scutellariae Radix, 4 to 7 parts by weight of Asparagi Radix, 2 to 4 parts by weight of Chuanxiong Rhizoma, 1 to 3 parts by weight of Cervi Cornu Pantotrichum, and 1 to 3 parts by weight of Aconiti Lateralis Radix Praeparata.

18. The method as claimed in claim 17, wherein the Chinese medicinal composition includes: 8 to 10 parts by weight of Hedysari Radix, 3 to 5 parts by weight of Scutellariae Radix, 5 to 6 parts by weight of Asparagi Radix, 3 parts by weight of Chuanxiong Rhizoma, 2 parts by weight of Cervi Cornu Pantotrichum, and 2 parts by weight of Aconiti Lateralis Radix Praeparata.

19. The method as claimed in claim 10, wherein the dementia is selected from the group consisting of degenerative dementia, vascular dementia, and mixed dementia.

20. The method as claimed in claim 19, wherein the dementia is a degenerative dementia selected from the group consisting of Alzheimer's disease, dementia with Lewy body, Frontal-temporal dementia, Parkinson's disease dementia, and Huntington's disease.

21. The method as claimed in claim 20, wherein the dementia is Alzheimer's disease.

22. The method as claimed in claim 21, wherein treating dementia includes promoting neurogenesis.
